(19) United States
(12) Patent Application Publication
(10) Pub. No.: US 2021/0290047 A1
(43) Pub. Date: Sep. 23, 2021

(54) IMAGE PROCESSING APPARATUS, METHOD OF OPERATING IMAGE PROCESSING APPARATUS, AND NON-TRANSITORY COMPUTER READABLE RECORDING MEDIUM

(71) Applicant: OLYMPUS CORPORATION, Tokyo (JP)

(72) Inventors: Yusuke SUZUKI, Tokyo (JP); Atsushi CHIBA, Tokyo (JP); Takahiro IIDA, Tokyo (JP)

(73) Assignee: OLYMPUS CORPORATION, Tokyo (JP)

(21) Appl. No.: 17/340,342

(22) Filed: Jun. 7, 2021

Related U.S. Application Data
(63) Continuation of application No. PCT/JP2018/045358, filed on Dec. 10, 2018.

(51) Int. Cl. A61B 1/04; A61B 1/00; A61B 1/273

(52) U.S. Cl. CPC A61B 1/041; A61B 1/00045; A61B 1/2736; A61B 1/00009

(57) ABSTRACT

An image processing apparatus includes: a processor including hardware. The processor is configured to extract pieces of positional information associated with image data captured by a capsule endoscope introduced into a digestive tract to calculate a movement amount of an image capturing position of the capsule endoscope in a luminal direction, acquire, from the pieces of positional information, a change amount of the image capturing position of the capsule endoscope in a direction different from the luminal direction, select first position information from among the pieces of positional information, the first position information being position information for which the change amount of the positional information is smaller than a first threshold and the movement amount is smaller than a second threshold, and generate a shape model of the digestive tract by using the first position information.

### CROSS-REFERENCE TO RELATED APPLICATION

This application is a continuation of International Application No. PCT/JP2018/045358, filed on Dec. 10, 2018, the entire contents of which are incorporated herein by reference.

### BACKGROUND

### 1. Technical Field

The present disclosure relates to an image forming apparatus, a method of operating the image forming apparatus, and a non-transitory computer readable recording medium.

### 2. Related Art

In the relate art, a technique for observing a subject by using a series of images that are acquired by a capsule endoscope by sequentially capturing images of inside a digestive tract of the subject has been known (for example, see Japanese Patent No. 5248834).

### SUMMARY

In some embodiments, an image processing apparatus includes: a processor comprising hardware. The processor is configured to extract pieces of positional information associated with image data captured by a capsule endoscope introduced into a digestive tract to calculate a movement amount of an image capturing position of the capsule endoscope in a luminal direction, acquire, from the pieces of positional information, a change amount of the image capturing position of the capsule endoscope in a direction different from the luminal direction, select first position information from among the pieces of positional information, the first position information being position information for which the change amount of the positional information is smaller than a first threshold and the movement amount is smaller than a second threshold, and generate a shape model of the digestive tract by using the first position information.

In some embodiments, provided is a method of generating a shape model of a digestive tract. The method includes: extracting pieces of positional information associated with image data captured by a capsule endoscope introduced into a digestive tract to calculate a movement amount of an image capturing position of the capsule endoscope in a luminal direction; acquiring, from the pieces of positional information, a change amount of the image capturing position of the capsule endoscope in a direction different from the luminal direction; selecting first position information from among the pieces of positional information, the first position information being position information for which the change amount of the positional information is smaller than a first threshold and the movement amount is smaller than a second threshold; and generating a shape model of the digestive tract by using the first position information.

In some embodiments, provided is a non-transitory computer readable recording medium with an executable program recorded thereon. The program causes a processor, which an image processing apparatus had, to execute extracting pieces of positional information associated with image data captured by a capsule endoscope introduced into a digestive tract to calculate a movement amount of an image capturing position of the capsule endoscope in a luminal direction; acquiring, from the pieces of positional information, a change amount of the image capturing position of the capsule endoscope in a direction different from the luminal direction; selecting first position information from among the pieces of positional information, the first position information being position information for which the change amount of the positional information is smaller than a first threshold and the movement amount is smaller than a second threshold; and generating a shape model of the digestive tract by using the first position information.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an endoscope system including an image processing apparatus according to a first embodiment of the present disclosure;

FIG. 2 is a block diagram illustrating a state in which a recording medium illustrated in FIG. 1 is connected to the image processing apparatus;

FIG. 3 is a diagram illustrating an example of an in-vivo image;

FIG. 4 is a diagram illustrating an example of an in-vivo image;

FIG. 5 is a flowchart illustrating operation of the image processing apparatus illustrated in FIG. 2;

FIG. 6 is a diagram illustrating how a model generation unit generates a shape model of a digestive tract;

FIG. 7 is a diagram illustrating how a model generation unit generates a shape model of a digestive tract;

FIG. 8 is a diagram illustrating how a model generation unit generates a shape model of a digestive tract;

FIG. 9 is a diagram illustrating how the model generation unit generates the shape model of the digestive tract; and

FIG. 10 is a diagram illustrating how a display control unit of a fourth modification causes a display device to display a shape model of a digestive tract.

### DETAILED DESCRIPTION

Embodiments of an image processing apparatus, a method of operating the image processing apparatus, and a non-transitory computer readable recording medium according to the present disclosure will be described below with reference to the drawings. The present disclosure is not limited by the embodiments below. The present disclosure is applicable to a general image processing apparatus that performs image processing on an image that is captured by a capsule endoscope inside a digestive tract of a subject, a method of operating the image processing apparatus, and a computer readable recording medium storing therein a program for operating the image processing apparatus.

Further, in the description of the drawings, the same or corresponding components are appropriately denoted by the same reference symbols. Furthermore, it is necessary to note that the drawings are schematic, and dimensional relations among the components, ratios among the components, and the like may be different from the actual ones. Moreover, the drawings may include portions that have different dimensional relations or ratios.

### First Embodiment

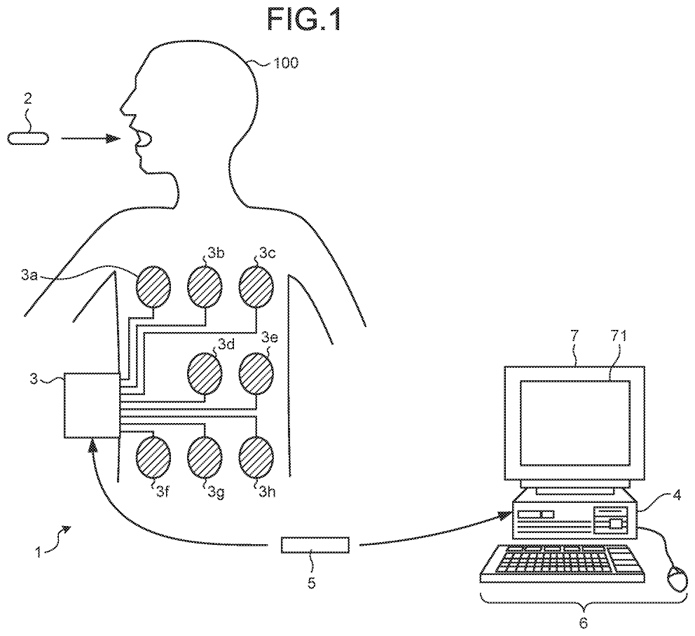

FIG. 1 is a schematic diagram illustrating an endoscope system including an image processing apparatus according to a first embodiment of the present disclosure. An endoscope system 1 is a system that uses a capsule endoscope 2 as a swallow-type medical device to capture in-vivo images inside a subject 100, and allows a doctor or the like to observe the in-vivo images. As illustrated in FIG. 1, the endoscope system 1 includes, in addition to the capsule endoscope 2, a receiving device 3, an image processing apparatus 4, a portable recording medium 5, an input device 6, and a display device 7.

The recording medium 5 is a portable recording medium for transferring data between the receiving device 3 and the image processing apparatus 4, and is configured so as to be removably attachable to each of the receiving device 3 and the image processing apparatus 4.

The capsule endoscope 2 is a capsule type endoscope device with a size that can be introduced into an organ of the subject 100, is introduced into the organ of the subject 100 by being ingested or the like, moves inside the organ by peristaltic movement or the like, and sequentially captures in-vivo images. Further, the capsule endoscope 2 sequentially transmits image data generated by image capturing.

The receiving device 3 includes a plurality of receiving antennas 3a to 3h, and receives the image data from the capsule endoscope 2 inside the subject 100 by using at least one of the receiving antennas 3a to 3h. Further, the receiving device 3 stores the received image data in the recording medium 5 that is mounted in the receiving device 3. Meanwhile, the receiving antennas 3a to 3h may be arranged on a body surface of the subject 100 as illustrated in FIG. 1, or may be arranged on a jacket to be worn by the subject 100. Furthermore, it is sufficient for the receiving device 3 to include one or more receiving antennas, and the number of the receiving antennas is not specifically limited to eight.

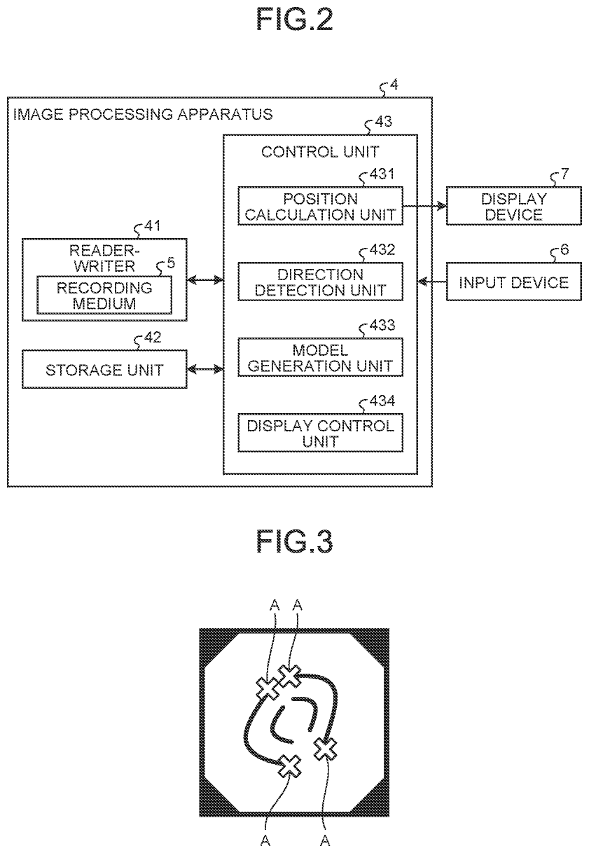

FIG. 2 is a block diagram illustrating a state in which the recording medium illustrated in FIG. 1 is connected to the image processing apparatus. As illustrated in FIG. 2, the image processing apparatus 4 includes a reader-writer 41, a storage unit 42, and a control unit 43.

The reader-writer 41 has a function as an image acquisition unit that acquires image data as a processing target from outside. Specifically, when the recording medium 5 is mounted in the reader-writer 41, the reader-writer 41 loads image data (an in-vivo image group including a plurality of in-vivo images that are captured (acquired) in chronological order by the capsule endoscope 2) stored in the recording medium 5, under the control of the control unit 43. Further, the reader-writer 41 transfers the loaded in-vivo image group to the control unit 43. Furthermore, the in-vivo image group transferred to the control unit 43 is stored in the storage unit 42.

The storage unit 42 stores therein the in-vivo image group that is transferred from the control unit 43. Further, the storage unit 42 stores therein various programs (including a program for operating the image processing apparatus) executed by the control unit 43, information needed for a process performed by the control unit 43, and the like. The storage unit 42 is implemented by various integrated circuit (IC) memories, such as a flash memory, a read only memory (ROM), and a random access memory (RAM), a built-in hard disk, a hard disk that is electrically connected by a data communication terminal, or the like.

The control unit 43 is configured with a general processor, such as a central processing unit (CPU), a dedicated processor for various arithmetic circuits, such as an application specific integrated circuit (ASIC), that implement specific functions. The control unit 43 reads the program (including the program for operating the image processing apparatus) stored in the storage unit 42, and controls entire operation of the endoscope system 1 in accordance with the program. As illustrated in FIG. 2, the control unit 43 includes a position calculation unit 431, a direction detection unit 432, a model generation unit 433, and a display control unit 434.

The position calculation unit 431 calculates positional information indicating a position at which each of the images is captured by the capsule endoscope 2 introduced in the digestive tract. Specifically, the position calculation unit 431 calculates positional information indicating a position at which each of the images is captured on the basis of intensity of a signal that each of the receiving antennas 3a to 3h has received from the capsule endoscope 2. Further, the position calculation unit 431 calculates a difference between pieces of positional information on chronologically successive images to calculate a change amount of the positional information between the images. The position calculation unit 431 may calculate the positional information on the capsule endoscope 2 by causing a magnetic field detection unit arranged outside the subject 100 to detect a magnetic field generated by a magnetic field generation unit that is arranged inside the capsule endoscope 2.

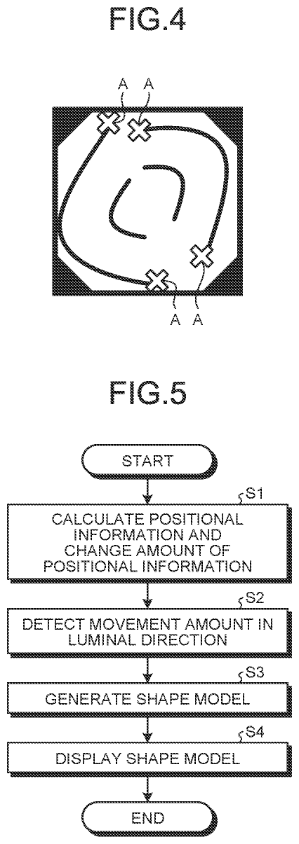

The direction detection unit 432 detects a movement amount of the capsule endoscope 2 in a luminal direction when the capsule endoscope 2 captures each of the images. FIG. 3 and FIG. 4 are diagrams illustrating an example of in-vivo images. FIG. 3 and FIG. 4 illustrate chronologically successive images. As illustrated in FIG. 3, the direction detection unit 432 sets feature points A in the image. The feature points A are, for example, end portions of a ruge or concavity and convexity in the digestive tract. If the feature points A are moved as illustrated in FIG. 4 from the state as illustrated in FIG. 3, the direction detection unit 432 detects a movement amount of the capsule endoscope 2 in the luminal direction on the basis of movement amounts of the feature points A. The direction detection unit 432 may detect the movement amount of the capsule endoscope 2 in the luminal direction by calculating similarity or the like between chronologically successive images. Further, the direction detection unit 432 may detect the movement amount of the capsule endoscope 2 in the luminal direction on the basis of information that is detected by a sensor, such as an acceleration sensor, mounted on the capsule endoscope 2.

The model generation unit 433 selects first position information from among the pieces of positional information on the basis of the change amount of the positional information and the movement amount in the luminal direction, and generates a shape model of the digestive tract by using the first position information. Specifically, the model generation unit 433 selects the first position information from among the pieces of positional information, the first position information being position information for which the change amount of the positional information is smaller than a first threshold and the movement amount in the luminal direction is smaller than a second threshold. Meanwhile, the first threshold and the second threshold are adequately small values such that the positional information is not affected by the peristaltic movement of the digestive tract, and may be predetermined or may be set by a user such as a doctor. Further, the first threshold and the second threshold may be variable depending on image capturing conditions such as a frame rate of the capsule endoscope 2.

The display control unit 434 performs predetermined image processing on the images stored in the storage unit 42, performs a predetermined process, such as data decimation or a gradation process, in accordance with an image display range of the display device 7, and thereafter causes the display device 7 to display a representative image.

The input device 6 is configured with a keyboard, a mouse, and the like, and receives operation performed by the user.

The display device 7 is configured with a liquid crystal display or the like, and displays images including the representative image under the control of the display control unit 434.

Next, a process of generating the shape model by the image processing apparatus 4 will be described. FIG. 5 is a flowchart illustrating operation of the image processing apparatus illustrated in FIG. 2. As illustrated in FIG. 5, the position calculation unit 431 calculates positional information indicating a position at which each of the images of the in-vivo image group is captured by the capsule endoscope 2, and further calculates the change amount of the positional information between the images (Step S1).

Subsequently, the direction detection unit 432 detects the movement amount of the capsule endoscope 2 in the luminal direction when each of the images of the in-vivo image group is captured (Step S2).

Then, the model generation unit 433 selects the first position information from among the pieces of positional information on the basis of the change amount of the positional information and the movement amount in the luminal direction, and generates the shape model of the digestive tract by using the selected first position information (Step S3).

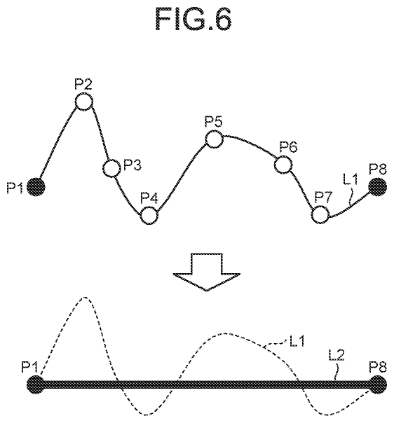

FIG. 6 is a diagram illustrating how the model generation unit generates the shape model of the digestive tract. As illustrated in FIG. 6, it is assumed that the model generation unit 433 selects pieces of positional information P1 and P8 as the first position information from among pieces of positional information P1 to P8. Then, the model generation unit 433 generates the shape model of the digestive tract by using the pieces of positional information P1 and P8.

If the shape model is generated by using the pieces of positional information P1 to P8, a curved shape that is not actually present appears due to the influence of the peristaltic movement of the digestive tract, so that a curve as represented by a line L1 is obtained. In contrast, the model generation unit 433 is able to generate a line L2 in which the influence of the peristaltic movement of the digestive tract is reduced by connecting between the pieces of positional information P1 and P8, which are the first position information, by a straight line, for example.

Thereafter, the display control unit 434 causes the display device 7 to display the shape model of the digestive tract generated by the model generation unit 433 (Step S4), and a series of processes is terminated.

As described above, according to the first embodiment, the model generation unit 433 generates the shape model by using the first position information that is acquired when the change amount of the positional information and the movement amount in the luminal direction are adequately reduced, so that it is possible to generate the shape model of the digestive tract in which the influence of the peristaltic movement of the digestive tract is reduced.

First Modification

In a first modification, the model generation unit 433 corrects second position information, which is the pieces of positional information excluding the first position information, to position information that represents a position on the shape model. Specifically, the model generation unit 433 corrects the second position information to the position information that represents the position on the shape model, on the basis of a ratio of a distance between adjacent pieces of the first position information to a distance between adjacent pieces of the positional information in order of image capturing.

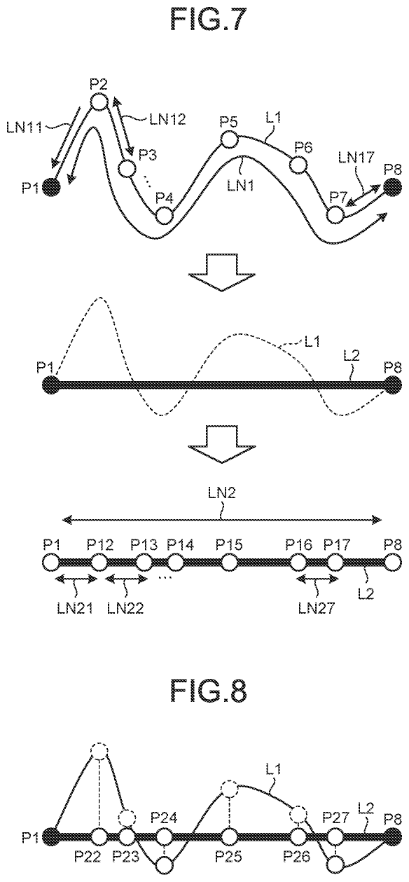

FIG. 7 is a diagram illustrating how a model generation unit of the first modification generates a shape model of a digestive tract. As illustrated in FIG. 7, the model generation unit 433 calculates a distance LN1 between cumulative adjacent pieces of the first position information on the line L1 and further calculates distances LN11 to LN17 where each distance is a distance between adjacent pieces of the positional information. The distance LN1 is the distances LN11 to LN17 (LN1=LN11+LN12+ . . . +LN17). Further, the model generation unit 433 calculates a distance LN2 between adjacent pieces of the first position information on the line L2. Subsequently, the model generation unit 433 corrects the pieces of second position information P2 to P7 to pieces of position information that represent positions on the shape model. Specifically, the model generation unit 433 corrects the pieces of second position information P2 to P7 to pieces of second position information P12 to P17 such that a relationship of LN21=(LN11/LN1)×LN2, LN22=(LN12/LN1)×LN2, . . . , LN27=(LN17/LN1)×LN2 is satisfied.

As described above, according to the first modification, it is possible to recognize a position on the shape model even for an image that corresponds to the second position information other than the first position information.

Second Modification

In a second modification, the model generation unit 433 corrects the second position information to position information that represents a position projected on the shape model.

FIG. 8 is a diagram illustrating how a model generation unit of the second modification generates the shape model of the digestive tract. As illustrated in FIG. 8, the model generation unit 433 corrects the pieces of second position information P2 to P7 to pieces of second position information P22 to P27 projected on the shape model. Specifically, the model generation unit 433 projects each piece of the second position information P2 to P7 on the line L2 in a direction perpendicular to the line L2, thereby correcting the pieces of second position information P2 to P7 to the pieces of second position information P22 to P27.

Third Modification

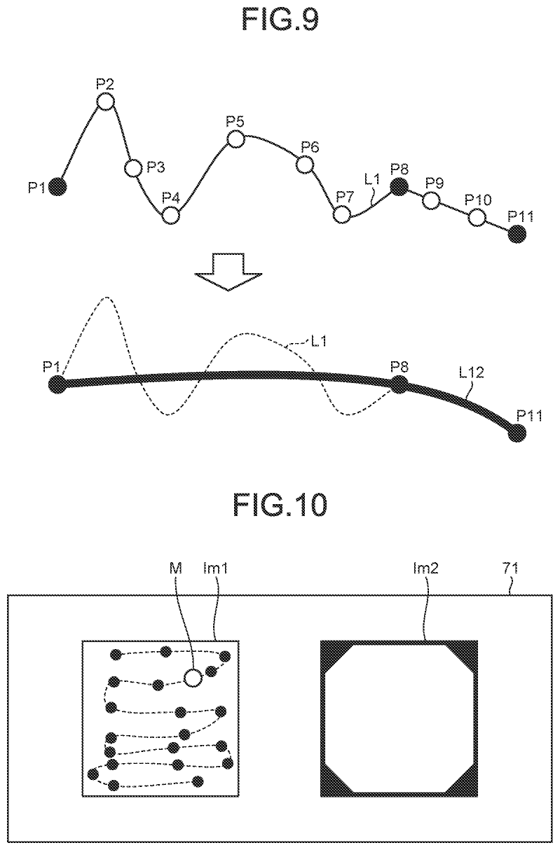

FIG. 9 is a diagram illustrating how a model generation unit of a third modification generates a shape model of a digestive tract. As illustrated in FIG. 9, the model generation unit 433 may perform fitting on pieces of first position information P1, P8, and P11 by a curve L12. Specifically, the model generation unit 433 is able to calculate the curve L12 that smoothly connects between the pieces of first position information P1, P8, and P11 by a spline function.

Fourth Modification

FIG. 10 is a diagram illustrating a state in which a display control unit of a fourth modification causes a display device to display a shape model of a digestive tract. As illustrated in FIG. 10, the display control unit 434 may display an image Im1 that represents a shape model generated by the model generation unit 433 and an in-vivo image Im2 that corresponds to a mark M in the shape model, on a screen 71 of the display device 7.

According to one aspect of the present disclosure, it is possible to provide an image processing apparatus, a method of operating the image processing apparatus, and a non-transitory computer readable recording medium storing a program for operating the image processing apparatus, which are able to generate a shape model of a digestive tract in which an influence of peristaltic movement of a digestive tract is reduced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising:
a processor comprising hardware, the processor being configured to
extract pieces of positional information associated with image data captured by a capsule endoscope introduced into a digestive tract to calculate a movement amount of an image capturing position of the capsule endoscope in a luminal direction,
acquire, from the pieces of positional information, a change amount of the image capturing position of the capsule endoscope in a direction different from the luminal direction,
select first position information from among the pieces of positional information, the first position information being position information for which the change amount of the positional information is smaller than a first threshold and the movement amount is smaller than a second threshold, and
generate a shape model of the digestive tract by using the first position information.

2. The image processing apparatus according to claim 1, wherein the processor is further configured to correct second position information, which is the pieces of positional information excluding the first position information, to position information that represents a position on the shape model.

3. The image processing apparatus according to claim 2, wherein the processor is further configured to correct the second position information to position information that represents a position on the shape model, based on a ratio of a distance between adjacent pieces of the first position information to a distance between adjacent pieces of the positional information in order of image capturing.

4. The image processing apparatus according to claim 2, wherein the processor is further configured to correct the second position information to position information that represents a position projected on the shape model.

5. A method of generating a shape model of a digestive tract, the method comprising:
extracting pieces of positional information associated with image data captured by a capsule endoscope introduced into a digestive tract to calculate a movement amount of an image capturing position of the capsule endoscope in a luminal direction;
acquiring, from the pieces of positional information, a change amount of the image capturing position of the capsule endoscope in a direction different from the luminal direction;
selecting first position information from among the pieces of positional information, the first position information being position information for which the change amount of the positional information is smaller than a first threshold and the movement amount is smaller than a second threshold; and
generating a shape model of the digestive tract by using the first position information.

6. A non-transitory computer readable recording medium with an executable program recorded thereon, the program causing a processor, which an image processing apparatus had, to execute
extracting pieces of positional information associated with image data captured by a capsule endoscope introduced into a digestive tract to calculate a movement amount of an image capturing position of the capsule endoscope in a luminal direction;
acquiring, from the pieces of positional information, a change amount of the image capturing position of the capsule endoscope in a direction different from the luminal direction;
selecting first position information from among the pieces of positional information, the first position information being position information for which the change amount of the positional information is smaller than a first threshold and the movement amount is smaller than a second threshold; and
generating a shape model of the digestive tract by using the first position information.
